(19) United States
(12) Patent Application Publication
(10) Pub. No.: US 2009/0093709 A1
(43) Pub. Date: Apr. 9, 2009

(54) Noise reduction system and methods for magnetic resonance imaging

(75) Inventors: Anand S. Patel, Boston, MA (US); Philip M. Robson, Brookline, MA (US); Charles A. McKenzie, London (CA); Daniel K. Sodickson, Larchmont, NY (US)

Correspondence Address: WOLF GREENFIELD & SACKS, P.C., 600 ATLANTIC AVENUE, BOSTON, MA, 02210-2206, US

(73) Assignee: Beth Israel Deaconess Medical Center, Inc., Boston, MA (US)

(21) Appl. No.: 12/152,940

(22) Filed: May 19, 2008

Related U.S. Application Data
(60) Provisional application No. 60/930,774, filed on May 18, 2007.

(51) Int. Cl. A61B 5/055

(52) U.S. Cl. 600/411

(57) ABSTRACT

In one aspect, a method for noise reduction in parallel magnetic resonance (MR) imaging using a plurality of radio frequency (RF) coils is provided. The method comprises performing a first MR scan of a target to obtain first MR data, performing a second MR scan of the target to obtain second MR data, the second MR data obtained from operating the plurality of coils substantially in parallel, computing a noise estimate associated with the second MR data based at least in part on the first and second MR data, and obtaining a noise-reduced image based at least in part on the second MR data and the noise estimate.

### RELATED APPLICATIONS

This application claims the benefit under 35 U.S.C. §119(e) of U.S. Provisional Application 60/930,774, filed May 18, 2007, entitled “NOISE REDUCTION SYSTEM AND METHODS FOR MAGNETIC RESONANCE IMAGING,” which is herein incorporated by reference in its entirety.

### FIELD OF THE INVENTION

The present disclosure is directed to noise reduction in magnetic resonance (MR) imaging, and more particularly to reduction of acceleration-related noise in parallel MR.

### BACKGROUND

Magnetic resonance imaging (MRI) is a technique used frequently in medical settings to produce images of the inside of the human body. MRI is based on detecting nuclear magnetic resonance (NMR) signals, which are electromagnetic waves emitted by atomic nuclei in response to state changes resulting from applied electromagnetic fields. In particular, magnetic resonance techniques involve detecting NMR signals produced upon the re-alignment or relaxation of the nuclear spins of atoms in the tissue of the human body. MR techniques may be used to image and study the properties of tissue in a variety of regions of the human body, for example, for detection and/or diagnosis of tissue anomalies, study of blood flow, etc. The applied electromagnetic fields and detection of the resulting MR signals may be performed using a transmit and receiver radio frequency (RF) coil.

In the past, MRI data was obtained using a single transmit/receiver coil. As a result of having only a single MR detector, only one line of data could be acquired at a time. Recently, techniques for using multiple transmit/receiver coils have been developed that facilitate acquiring multiple lines of data simultaneously. MR techniques using multiple transmit and/or receive coils to image a subject is referred to herein as parallel MR. Parallel MR takes advantage of the spatial information available using an appropriately arranged array of RF coils. In parallel MR, some number of the RF coils in an array may be independently excited (e.g., RF power may be transmitted over independent channels to multiple respective RF coils) and/or independently measured (e.g., measurements may be obtained/received from multiple RF coils over respective independent channels).

Parallel MR has circumvented previous limits on speed and efficiency, effecting a reduction in image acquisition times due, in part, to the fact that multiple lines of data may be obtained simultaneously. As a result, parallel MR has become an integral technology in modern MRI clinical and research scanners. However, an important trade-off exists in conventional parallel MR. Specifically, the acceleration of data acquisition in parallel MR results in a corresponding decrease in signal-to-noise ratio (SNR). In general, n receiver coils may be capable of achieving an n-fold reduction in scan time. The amplification of noise in parallel image reconstruction, however, also increases with the reduction in scan time. The term “acceleration-related noise” refers herein to noise associated with accelerated data acquisition and reconstruction in parallel MR (also referred to as geometry factor, or g-factor, noise in the literature).

Acceleration-related noise results, in part, from overlap or crosstalk between the various coils in a multiple coil array. That is, acceleration-related noise is produced when the coil array does not provide sufficiently independent data. Conventional attempts to reduce acceleration-related noise have generally been unsatisfactory for clinical parallel MRI. For example, one approach to reducing acceleration-related noise includes approximating the acceleration-related noise using an exhaustive search method, and subtracting the approximated noise from the reconstructed pixel values (Larkman et al., “Beyond the g-factor limit in sensitivity encoding using joint histogram entropy,” Magnetic Resonance in Medicine, 55(1): 153-160, 2005). However, such a search may take hours to perform (e.g., up to 18 hours for an exhaustive search), which substantially negates the benefits of using parallel MR in the first place (e.g., relatively long computation times counteract the reduction in scan time achievable using parallel MR).

### SUMMARY OF INVENTION

Some embodiments include a method for noise reduction in parallel MR imaging using a plurality of RF coils, the method comprising, performing a first MR scan of a target to obtain first MR data, performing a second MR scan of the target to obtain second MR data, the second MR data obtained from operating the plurality of coils substantially in parallel, computing a noise estimate associated with the second MR data based at least in part on the first and second MR data, and obtaining a noise-reduced image based at least in part on the second MR data and the noise estimate.

Some embodiments include a computer storage device encoded with a program for execution on at least one processor, the program when executed performs a method for noise reduction in parallel MR imaging, the method comprising acts of receiving first MR data, receiving second MR data obtained from operating a plurality of RF coils substantially in parallel, computing a noise estimate associated with the second MR data based at least in part on the first and second MR data, and computing a noise-reduced image based at least in part on the second MR data and the noise estimate.

Some embodiments include a system for performing parallel MR imaging comprising a scanning apparatus comprising a plurality of RF coils, the scanning apparatus configured to perform accelerated image acquisition by operating the plurality of coils substantially in parallel to obtain parallel MR data, and a signal processing device programmatically coupled with the scanning apparatus to obtain the parallel MR data from the scanning apparatus, the signal processing device configured to reduce acceleration-related noise in the parallel MR data by receiving reference MR data, computing a noise estimate associated with the parallel MR data based at least in part on the parallel MR data and the reference MR data, and generating at least one noise-reduced MR image based at least in part on the accelerated MR data and the noise estimate.

### BRIEF DESCRIPTION OF DRAWINGS

FIG. 1 illustrates a method for noise reduction in parallel MR imaging using a plurality of RF coils, in accordance with some embodiments of the invention;

FIG. 2 illustrates in vivo, T1-weighted brain images obtained with an 8 element head array, the upper left image showing an unaccelerated gold standard, the upper middle showing a 2×4-fold accelerated Sensitivity Encoding (SENSE) reconstruction, the upper right showing a SENSE g-factor noise map, the lower left showing a low resolution reference image, the lower middle showing a least squares noise reduction (LSNR) reconstruction, and the lower right showing an LSNR g-factor noise map, in accordance with some embodiments of the invention;

FIG. 3 illustrates in vivo, T2-weighted brain images obtained with an 8 element head array, the upper left showing an unaccelerated gold standard, the upper middle showing a 2×4-fold accelerated SENSE reconstruction, the upper right showing a SENSE g-factor noise map, the lower left showing a low resolution reference image, the lower middle showing an LSNR reconstruction, and the lower right showing an LSNR g-factor noise map, in accordance with some embodiments of the invention;

FIG. 4 is a table comparing artifact power (AP) and g-factor noise among various results obtained using noise reduction techniques, in accordance with some embodiments of the invention;

FIG. 5 illustrates in vivo T2-weighted brain images obtained with an 8 element head array, the left showing a 2×4-fold accelerated SENSE reconstruction with 4 lesions added, and right showing an LSNR reconstruction, in accordance with some embodiments of the invention;

FIG. 6 illustrates in vivo T2-weighted brain images obtained with an 8 element head array, the upper left showing an unaccelerated gold standard, the upper middle showing 4-fold accelerated SENSE reconstruction, the upper right showing a SENSE g-factor noise map, the lower left showing a low resolution reference image, the lower middle showing an LSNR reconstruction, and the lower right showing an LSNR g-factor noise map, in accordance with some embodiments of the invention;

FIG. 7 illustrates in vivo T1-weighted brain images obtained with an 8 element head array, the upper left showing the unaccelerated gold standard, the upper middle showing a 2×4-fold accelerated masked SENSE reconstruction, the upper right showing a SENSE g-factor noise map, the lower left showing a low resolution reference image, the lower middle showing an LSNR reconstruction, and the lower right showing an LSNR g-factor noise map, in accordance with some embodiments of the invention;

FIG. 8 illustrates in vivo T1-weighted brain image obtained with an 8 element head array, the upper left showing the unaccelerated gold standard, the upper middle showing a 2×4-accelerated masked SENSE reconstruction from FIG. 7, the upper right showing a SENSE g-factor noise map from FIG. 7, the lower left showing an LSNR hybrid reconstruction, where the pericalvarial tissue is from the masked SENSE image, while the remaining parenchyma is derived from an LSNR reconstruction, and the lower right shows an LSNR hybrid g-factor noise map, in accordance with some embodiments of the invention;

FIG. 9 illustrates phantom data obtained with a 32-element (4×4×2) body array, the upper left showing the unaccelerated gold standard, the upper right showing a 8×4-fold accelerated SENSE reconstruction, the lower left showing a 1-component least squares noise reduction, and the lower right showing a 9-component least-squares noise reduction, in accordance with some embodiments of the invention; and

FIG. 10 illustrates conditional number threshold (CNT) versus artifact power (AP) plots.

### DETAILED DESCRIPTION

As discussed above, conventional methods of reducing the effects of acceleration-related noise may be unsatisfactory in that they counteract the benefits of performing parallel MR. That is, the acquisition speed-ups may be nullified by the computational expense incurred in approximating the acceleration-related noise. Applicant has appreciated that a computationally inexpensive algorithm (e.g., an algorithm that can be performed in real-time during a scan) for reducing acceleration-related noise in parallel MR would facilitate the adoption of noise reduction algorithms in clinical settings by reducing the wait time for a clinician to have the data and begin making a diagnosis, reducing the time required for patients to be in the MR scanner (during which time a patient may move appreciably, and/or may become claustrophobic).

Various aspects of the invention derive from Applicant's recognition that a relatively low resolution reference image may be used to approximate a noiseless image of a target being scanned at accelerated acquisition rates. This approximation of a noiseless image may then be used to estimate acceleration-related noise without having to perform an exhaustive and/or computationally expensive search. The estimated acceleration-related noise may then be subtracted from the reconstructed pixel values to reduce the effects of acceleration-related noise. According to some embodiments, the noise reduction process described above may be performed in real-time or substantially in real-time. According to some embodiments, the noise reduction process described above may be performed on the order of one to ten seconds.

Concepts related to methods and apparatus according to the invention, and various embodiments thereof, are described below in greater detail. It should be appreciated that various aspects of the invention described herein may be implemented in any of numerous ways, and examples of specific implementations are provided herein for illustrative purposes only. For instance, while the exemplary embodiments presented below are based on Cartesian-type sampling, concepts and techniques of the invention may be used in conjunction with other sampling trajectories, such as radial and spiral trajectories.

Furthermore, aspects of the invention may be used in conjunction with both Fourier-based and non-Fourier-based methods for parallel MR. In a Fourier-based method, raw NMR data are stored in a data matrix called the k-space matrix, from which an image of the scanned target is generated by Fourier transformation. Essentially, Fourier transformation converts frequency information associated with NMR emissions detected by the RF coils to spatial information representing the distribution of nuclear spins in the scanned target. One class of Fourier based methods includes applying a reconstruction algorithm after Fourier transformation has been applied to the raw NMR data. Sensitivity encoding (SENSE) is an example of reconstruction in the image space.

According to the SENSE parallel imaging method and its variants, an aliased image (that is, a K-fold reduced image, where K is the acceleration/reduction factor) is obtained by Fourier transformation on the k-space. Each pixel in this aliased image is “unfolded” into K pixels that are equidistantly distributed in the desired full image. The process of unfolding involves a so-called sensitivity matrix (also know as a reconstruction matrix), which captures spatial sensitivities of the RF coils according to the particular geometric configuration at which scanning is performed. A sensitivity matrix may be calculated theoretically based on coil geometry, or derived by a calibration protocol carried out on a phantom image or on a pre-scan at the time of in vivo imaging. Methods for building a sensitivity matrix are well known in the art of parallel MR (for instance, in Pruessmann et al., “SENSE: Sensitivity Encoding for Fast MRI,” Magnetic Resonance in Medicine, 42(5): 952-962, 1999).

The SENSE reconstruction computation may be summarized as:

|X [symbol] =C⁻¹|A [symbol] (1)

where |X [symbol] is a complex vector representing the pixel intensities of the unfolded image, C is the sensitivity matrix, and |A [symbol] is a complex vector representing the pixel intensities of the folded image.

As discussed above, accelerated data acquisition and the corresponding image reconstruction process in parallel MR leads to acceleration-related noise in the reconstructed image. Thus, the vector |A [symbol] may be expressed in terms of a noiseless image with the addition of acceleration-related noise:

|A [symbol] =|A′ [symbol] +|dA [symbol] (2),

where |A′ [symbol] represents true pixel intensities in the folded image and |dA [symbol] represents the noise contribution. Similarly, the vector |X [symbol] may be expressed in terms of a noiseless image with the addition of acceleration-related noise:

|X [symbol] =|X′ [symbol] +|dX [symbol] (3),

where |X′ [symbol] represents true pixel intensities in the unfolded image and |dX [symbol] represents the noise contribution. The noise contribution |dX [symbol] may correspond to undesirable artifact in the unfolded image, which may negatively impact the applicability of parallel MR in clinical settings. There is a need to identify or approximate the noise contribution |dX [symbol] and to remove |dX [symbol] from |X [symbol] , so as to obtain a good approximation of the noiseless image |X′ [symbol] .

Substituting equations 2 and 3 into equation 1 yields:

|X [symbol] +|dX [symbol] =C⁻¹(|A′ [symbol] +|dA [symbol] ) (4).

Applicant has recognized that, due to the linear nature of the matrix operations involved, equation 4 can be rewritten as:

|dX [symbol] =C⁻¹|dA [symbol] (5).

In other words, |dX [symbol] , which represents noise in the unfolded image, can be viewed as the result of unfolding the noise contribution |dA [symbol] in the folded image.

Recall that the sensitivity matrix C is based on the configuration and geometry of the particular coil array from which the NMR data is acquired. Using singular value decomposition (SVD), the sensitivity matrix C may be factorized into principal components, yielding,

C=USV⁺ (6).

Accordingly, the inverse of the sensitivity matrix may be written as,

C⁻¹=VS⁻¹U⁺=Σₖ|vₖ [symbol] sₖ⁻¹ [symbol] uₖ| (7),

where k runs from 1 to the acceleration factor K (for example, K is the number of RF coils in the coil array), |vₖ [symbol] is the k-th column vector of the matrix V, [symbol] uₖ| is the k-th row vector of the matrix U⁺, sₖ is the k-th diagonal element of the diagonal matrix S. Performing appropriate substitutions in equations 3, 5 and 7 yields,

|X′ [symbol] =|X [symbol] −Σₖ|vₖ [symbol] sₖ¹ [symbol] uₖ|dA [symbol] (8).

Equation 8 expresses the noiseless image |X [symbol] in terms of the decomposed sensitivity matrix and the noise contribution |dA [symbol] in the folded image. Equation 8 can be rewritten as,

|X′ [symbol] =|X [symbol] −Σₖ|vₖ [symbol] sₖ⁻¹δAₖ (9),

where δAₖ is an unknown scalar value defined by the inner product of [symbol] uₖ| with the unknown noise contribution vector |dA [symbol] .

It has been appreciated that at high accelerations (i.e., high values of K), the sensitivity matrix C may become ill-conditioned. As a result, the inverse of the sensitivity matrix (i.e., C⁻¹) is dominated by the first few singular values and the associated singular vectors. For instance, at high acceleration, an ill-conditioned sensitivity matrix may be dominated by the most significant singular vector (i.e., the dominant eigenvector). As a result, the sensitivity matrix may be adequately described using the first singular value and the associated singular vector. Using this insight, equation 9 may be rewritten as,

|X′ [symbol] ≈|X [symbol] −|v₁ [symbol] s₁⁻¹δA₁ (10).

Note that equation 10 includes two unknowns, the noiseless image |X′ [symbol] and the scalar value δA₁, and therefore cannot be solved directly. It has been proposed that a search may be conducted to determine the scalar value δA₁. More specifically, a search algorithm may exhaustively test every value in a search domain and return a value for δA₁ that minimizes a selected metric, for instance, the joint entropy between the reconstructed image and a reference image (Larkman et al., “Beyond the g-factor limit in sensitivity encoding using joint histogram entropy,” Magnetic Resonance in Medicine, 55(1): 153-160, 2005). However, as discussed above, such searches are computationally expensive and tend to nullify any reductions in scan time achieved by using parallel MR.

Applicant has appreciated that, instead of performing a computationally expensive search, a low resolution reference image may be suitable as an approximation of the noiseless image |X′ [symbol] for purposes of computing δA₁. Examples of potentially suitable reference images include low resolution images that are routinely obtained in MRI to calibrate the scanner and/or to tune the RF coils. Thus estimated, δA₁ may then be substituted into equation 10 to compute an approximation of the noiseless image |X [symbol] .

More specifically, using equations 3 and 10, the image noise vector |dx [symbol] can be expressed as,

|dX [symbol] ≈|v₁ [symbol] s₁⁻¹δA₁ (11).

Separately, a relatively low resolution reference image |X_{ref} [symbol] may “stand-in” for the noiseless image |X [symbol] in equation 3, yielding,

|dX [symbol] ≈|X [symbol] −|X_{ref} [symbol] (12).

Combining equations 11 and 12 yields,

|X [symbol] −|X_{ref} [symbol] ≈|v₁ [symbol] s₁⁻¹δA₁ (13).

Equation 13 has only one unknown, namely δA₁, therefore a solution may be found algebraically. In some embodiments, equation 13 is solved using the Moore-Penrose least squares solution, thereby obtaining an estimate for δA₁. For example, the single dominant value δA₁ may be determined by,

δA₁≈s₁( [symbol] v₁|v₁ [symbol] )⁻¹ [symbol] v₁|(|X [symbol] −|X_{ref} [symbol] ) (14).

The determined value for δA₁ may then be substituted back into equation 10 to compute an approximation of the noiseless image |X′ [symbol] . As a result, an approximation for a noiseless image based on data obtained using parallel MR techniques (e.g., at a K-fold speed-up) may be directly computed without performing extensive or exhaustive searches. The term “directly computed” refers herein to determining the object of the computation using analytic methods that compute a single result for the object being computed. Solving for a variable in an expression wherein the resulting value of the variable is solved for once is an example of directly computing the value or result. Thus results obtained using iterative optimization schemes and other searching techniques that iteratively compute an estimate for the object being computed to find a result that minimizes or maximizes some metric or expression are not “directly computed” as used herein.

From a computational standpoint, searches may be more expensive relative to solving algebraic equations using, for example, least squares techniques. Thus, the speed benefits of parallel MR may be exploited without nullifying the acceleration with costly noise compensation algorithms. It should be appreciated that the sensitivity matrix C may be approximated using multiple singular values and the associated singular vectors, rather than just the first singular value and the associated singular vector. For instance, equation 9 may be rewritten alternatively as,

|X [symbol] ≈|X [symbol] −|v₁ [symbol] s₁⁻¹δA₁−|v₂ [symbol] s₂⁻¹δA₂−|v₃ [symbol] s₃⁻¹δA₃ (15).

The procedure described above for estimating δS₁ can be generalized to a procedure for simultaneously estimating multiple scalar values, such as δA₁, δA₂, and δA₃ in equation 15 above. This may be achieved by replacing |v [symbol] in the above procedure with a matrix containing a larger subset of the columns of V. In the case of equation 15, a matrix containing the first three columns of V may be used.

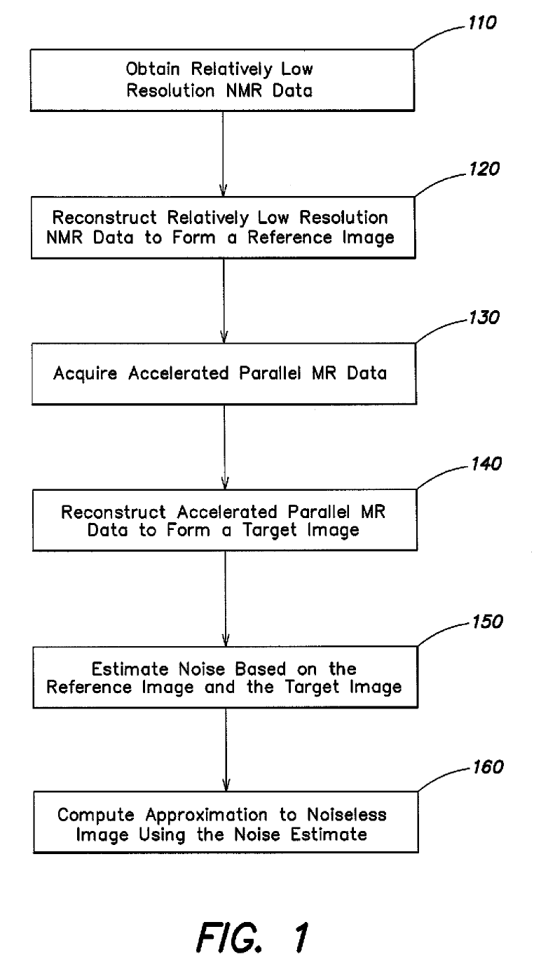

Accordingly, based on the Applicant's insights described above, a relatively low resolution reference image may be used to approximate acceleration-related noise, which can in turn be used to improve the quality of an image obtained using any one of various parallel MR techniques. FIG. 1 illustrates a method of reducing the effects of acceleration-related noise in parallel MR, in accordance with some embodiments of the invention.

In act 110, MR data is acquired at relatively low resolution. The MR data may be low resolution relative to the resolution of the MR data obtained to form the intended image of the target, for example, to form a clinical or diagnostic image of the target. Relatively low resolution MR data is routinely obtained during MRI procedures to calibrate the MR scanner (e.g., to calibrate the transmit/receive coils or otherwise tune the components of the scanner). This relatively low resolution MR data may then be reconstructed to form a reference image (act 120). As discussed above, reference images are often obtained for other purposes (e.g., calibration and/or tuning) and therefore may not increase the overall scan time of a typical MRI procedure, which often must be re-calibrated and/or retuned for each object that is scanned (e.g., because each object such as an individual human patient operates as a different load on the coil array).

In act 130, accelerated acquisition MR data is obtained from a coil array having multiple RF coils. For example, the coil array may include K coils and the MR data may be obtained at an K-fold reduction in acquisition time with respect to single coil acquisition. However, any amount of acceleration over single coil acquisition may be achieved, as the aspects of the invention are not limited in this respect. In general, the accelerated acquisition MR data is at a resolution appropriate for clinical use, such as for use in the detection of anomalous tissue, diagnostics, FMRI analysis, etc., although this is not a limitation on the aspects of the invention. The accelerated acquisition MR data may then be reconstructed into a target image (act 140). For example, the accelerated acquisition MR data may be reconstructed using the SENSE technique, or other appropriate parallel MR reconstruction techniques.

As discussed above, MR images reconstructed from accelerated acquisition MR data may be corrupted, at least somewhat, by acceleration-related noise. That is, the acceleration-related noise in parallel MR data may be reconstructed into image artifacts. In act 150, the acceleration-related noise is estimated based, at least in part, on the reference image and the target image. In some embodiments, the target image is reconstructed using a SENSE technique for parallel MR. In particular, the target image may be reconstructed using a sensitivity matrix associated with the coil array. This sensitivity matrix may be employed to facilitate estimating acceleration-related noise.

For example, the sensitivity matrix may be decomposed using one or more SVD techniques. The decomposed sensitivity matrix may then be used in connection with the target image and the reference image to estimate the acceleration-related noise, for example, as described in the derivation of equations provided in the foregoing. However, the reference image may be used in other ways in connection with the target image to estimate acceleration-related noise, as the aspects of the invention are not limited in this respect. In particular, Applicant's insight that the reference image may be used as an approximation of an image free of the effects of acceleration-related noise may be used in other ways to estimate the acceleration-related noise of the acquisition process.

In act 160, the estimated acceleration-related noise is used to correct the target image. For example, the estimated acceleration-related noise may be used to approximate artifacts in the target image resulting from the acceleration-related noise. There are numerous ways in which the acceleration-related noise may be used to approximate the resulting image artifacts. In some embodiments, the estimated acceleration-related noise may be used as an initial guess in a search for an optimally estimated acceleration-related noise. This estimated noise may then be subtracted from the target image.

In some embodiments, the computation cost of performing acts 150 and 160 may be insignificant relative to the acquisition time. That is, the speed-up in scan time achieved using parallel MR may not be substantially affected by the noise reduction computations. As such, noise introduced as a result of accelerated scan times using parallel MR may be, to some extent, compensated for without adding significant computation time that tends to negate parallel MR speed-ups using conventional techniques. In some embodiments, acts 150 and 160 may be performed substantially in real-time with little or no appreciable impact on the total time for image acquisition (e.g., noise compensation may be performed in a matter of seconds). However, noise compensation computations may be performed in any time frame, as the aspects of the invention are not limited in this respect.

It should be appreciated that the concepts and techniques described above may be used in conjunction with other methods for parallel MR, such as those that perform reconstructions in the k-space and those based on non-Fourier spatial distributions.

Furthermore, the various aspects of the invention described in the exemplary embodiments may be used alone or in any combination, and are not limited to the combinations explicitly described herein. Various techniques described herein may be used in connection with any MRI scanner and device that performs parallel MR acquisition, as the aspects of the invention are not limited for use with a particular MR device or apparatus.

Several experiments, both in vivo and on phantom objects, have been performed to study the effectiveness of noise reduction according to some embodiments of the invention. Hereinafter, various embodiments of the invention are referred to generically as Least Squares Noise Reduction (LSNR) algorithms/methods/techniques. It should be understood that the use of the generic term “LSNR” does not limit aspects of the invention to the details provided in the following description. It should also be understood that the invention is not limited to the particular software (e.g., software packages for data processing) and hardware (e.g. scanning equipments and computers) used in the experiments described below.

Various acceleration schemes are explored in these experiments and the performances are investigated for high numbers of RF coil elements. For the in vivo experiments, low resolution images obtained during scanning are used as reference images in the LSNR algorithms. These studies verify that the LSNR method does not replicate the reference image. The studies also verify that small lesions which are conspicuous in a high resolution image but not discernible in a lower resolution image are preserved after the application of an LSNR algorithm in which the lower resolution image is used as a reference image.

In some experiments, in vivo images were obtained by scanning brain tissues in human volunteers. Images were acquired on a 1.5-T GE Excite HD-x scanner (GE Healthcare, Waukesha, Wis.). Two-dimensional (2-D) axial images of the brain were acquired using a head array coil with 8 elements circumferentially distributed. Brain images were acquired with T₁-weighted (T₁-w) contrast by a spin echo sequence with a single signal average and with T₂-weighted (T₂-w) contrast by a fast spin echo sequence with an echo train length of 8 and with 2 signal averages. Acquisition parameters include: 22-cm field of view (FOV), 5-mm slice thickness, 256×256 image matrix, ±15.63-kHz acquisition bandwidth, repetition/echo time (TR/TE) for T₁-w of 500/20 ms and for T₂-w of 2500/85 ms. Images were acquired twice to allow a reference image to be obtained with independent noise content.

In some embodiments, low-resolution reference images were obtained from the center of the acquired k-space data and filtered with a Kaiser-Bessel window (β=2). Coil sensitivities for parallel imaging were obtained from the center of the image k-space in the manner of self-calibrated parallel imaging with the center of k-space filtered with the same Kaiser-Bessel window. Images were acquired fully sampled and later decimated with various acceleration factors. Brain images were maximally undersampled, with acceleration factor K equal to the number of coils n. Both undersampling in one direction and in two dimensions were investigated. In some experiments, the frequency encoded direction is undersampled after acquisition mimicking the case of a section from a volumetric image acquisition, for example, undersampling with K=K_{y}×K_{z} equal to 4×2 and 2×4. Discrete lesions were simulated by setting the pixel intensities of 4 small regions to unity in a single 2-D axial SENSE reconstructed image. In addition, some experiments explored the performance of the LSNR method with sub-maximal acceleration (K<n) with K=4. In some experiments, similar methods were performed for phantom studies, where a body array with 32 RF coil elements distributed as 4×4 anterior and posterior grids was used on a manufacturer's standard physiological phantom.

Images were reconstructed using the SENSE algorithm as described herein. Regularly undersampled data were reconstructed into aliased (i.e., folded) images by fast Fourier transform (FFT). Unfolding was then be achieved using the inverse of the sensitivity matrix found by SVD for groups of aliased pixels, as shown in equation 9. SENSE image reconstruction may be implemented both with and without coil sensitivity images mask, which may be constructed, for example, by determining the set of pixels that are less than 5% of the maximum reference image pixel intensity and by setting corresponding indices of the coil sensitivities to zero. G-Factor noise amplification may be reduced by eliminating aliased pixel positions from the reconstruction which are known to contribute no signal intensity from the scanned object.

In some embodiments, an LSNR algorithm was implemented on a pixel-by-pixel basis. The sensitivity matrix formed from the known coil sensitivities may be decomposed via SVD. The principal singular value and vector may then be used to find δA₁ with the vector of unfolded image pixels and the corresponding vector of pixels in the reference image. Multiple singular vectors could be used by finding noise-reduction vectors for the singular vectors corresponding to the multiple scaling parameters δAₖ, as indicated in equation 9.

For the original SENSE images, g-factor may be calculated according to well-known methods. For the LSNR method, noise reduction was quantified using a pseudo multiple replica method, in which many image replicas (e.g., 128) are reconstructed, each with artificial noise added to the acquired k-space. Image artifact power (AP) may also be calculated to quantitatively assess image quality. Artifact power is defined based on the differences of squared pixel values between the image of interest and an unaccelerated gold standard. More precisely,

$$AP = \frac{{\sum }_{i , j} \vert {\vert {I}_{i , j}^{unaccelarated} \vert}^{2} - {\vert {I}_{i , j}^{reconstructed} \vert}^{2} \vert}{{\sum }_{i , j} {\vert {I}_{i , j}^{unaccelarated} \vert}^{2}} . \qquad ( 16 )$$

AP is a measure of the amount of noise and artifact in an image, and it does not distinguish between these two types of image corruptions.

Associated with noise reduction is a potential increase in residual artifact. To ensure that artifact is not introduced without significant reduction in image noise, the artifact power for the LSNR image is calculated for maximal and submaximal accelerated in vivo reconstructions where a limit is placed on the condition number of the sensitivity matrix. Only groups of pixels for which the condition number of the sensitivity matrix is above a given threshold are operated on using the LSNR algorithm. Thus, by setting the threshold to a very large value, no pixels undergo noise reduction and the conventional SENSE image is produced; conversely, by setting the threshold to zero, all image pixels undergo noise reduction.

A major source of residual artifact in brain images is the pericalvarial tissue (PCT) which produces characteristic edge artifacts across the image. A scheme may be implemented to eliminate these artifacts from LSNR images by segmenting and removing the PCT from the SENSE reconstructed image before the LSNR algorithm is applied. This may be achieved by: automatically identifying the brain tissue without the PCT on the masked SENSE images, performing a LSNR algorithm on the remaining image, and then reintroducing the PCT to this reconstructed image.

Specifically, after performing the SENSE reconstruction with the masking technique described above, the ‘imextendedmin’ function in the Matlab Image Processing toolbox can be used to perform an extended minima transform on the masked SENSE image using a connectivity of 8 points to create a binary image differentiating the image (PCT and brain tissue) from non-tissue (image null space). The ‘bwlabel’ function with a pixel connectivity of 4 may then be applied to the binary image in order to classify the image structures based on their spatial orientation and connectivity. This function exploits the fact that the PCT in the image is surrounded on its exterior and interior by an area of low intensity that has greater than 4 point connectivity. The ‘bwlabel’ function results in an image where each connected part of the image is assigned a common integer value. By excluding the image null space outside of the image of interest, the brain tissue without the PCT may then be isolated before performing the LSNR algorithm on it, resulting in an LSNR image with only the brain tissue. Finally, using simple image addition, the PCT from the masked SENSE image may be reintroduced to this brain tissue LSNR image, resulting in a LSNR hybrid image. Using this method, the extent of PCT-related edge artifact in LSNR images can be determined.

Comparison of the appearances of noise-reduced images may be performed between images from the LSNR approach and from the joint entropy (JE) minimization search to assess the effectiveness of techniques disclosed herein and conventional exhaustive search techniques. JE reconstructions may be performed from coil sensitivity masked SENSE reconstructed images, with the same low-resolution reference image used in LSNR reconstructions. It should be noted that the JE method only converges to meaningful minima if the input SENSE and reference images are masked by coil sensitivity.

In comparison experiments, image reconstruction and processing was performed on a standard PC laptop computer with a 1.7 GHz processor and 2 GB RAM using the Matlab programming environment. In the noise reduction experiments, the LSNR algorithm took approximately 30 seconds to execute. By contrast, the corresponding JE minimization procedures took approximately 95 hours to complete. Thus, compared to JE minimization, LSNR is approximately four orders of magnitude faster in producing noise-reduced images.

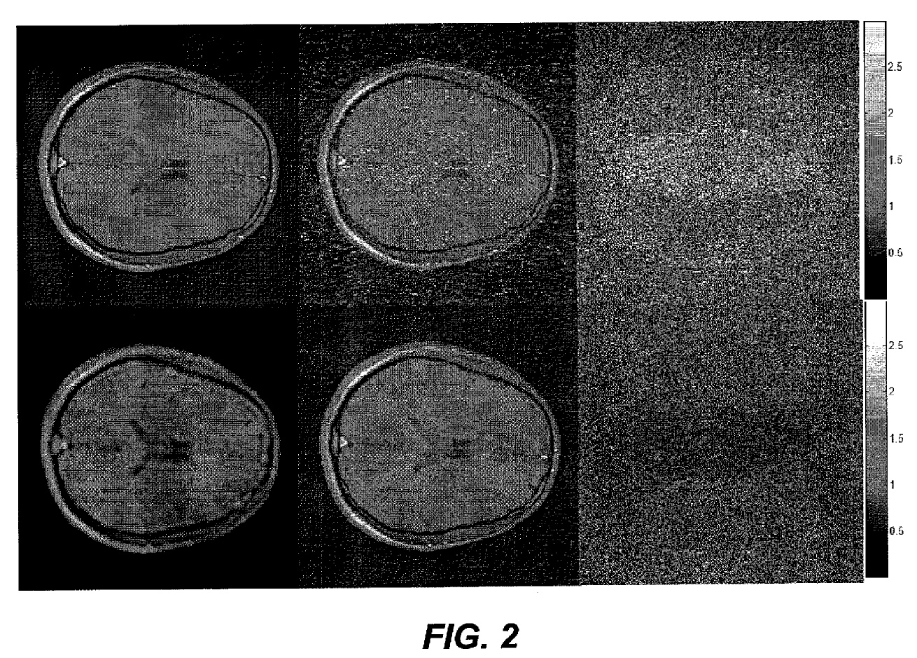

FIG. 2 illustrates T1-weighted in vivo brain images acquired with an 8 element head array and reconstructed at maximal acceleration of 2×4-fold. Significant noise reduction is achieved by the LSNR method without apparent direct replication of the reference image. The upper left image shows an unaccelerated gold standard, the upper middle shows a 2×4-fold accelerated SENSE reconstruction, the upper right shows a SENSE g-factor noise map, the lower left shows a low resolution reference image, the lower middle shows an LSNR reconstruction, and the lower right shows an LSNR g-factor noise map.

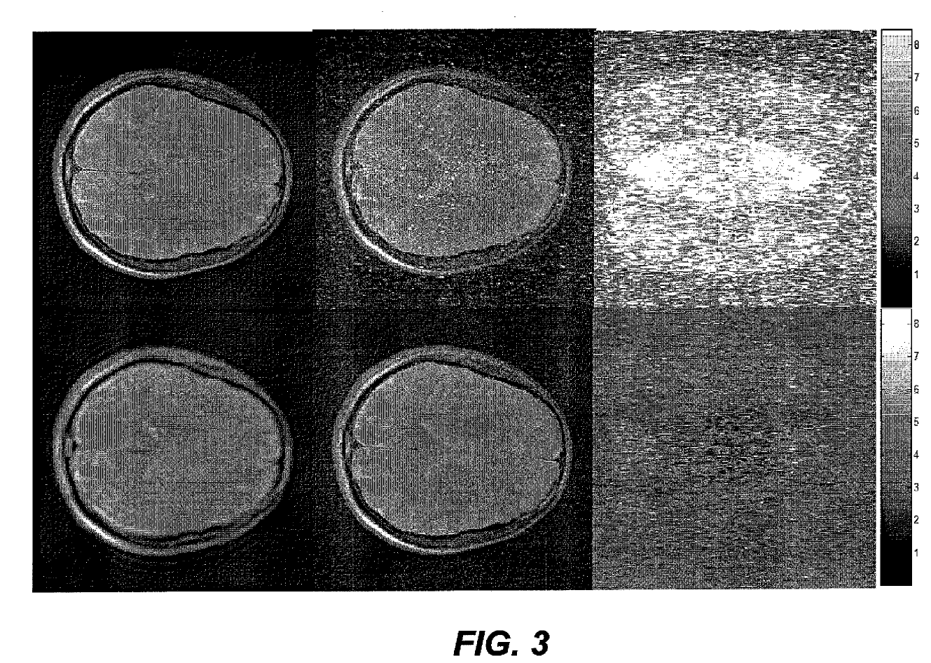

FIG. 3 illustrates the studies repeated in T2-weighted brain images obtained with an 8 element head array, showing similar results as in the T1-weighted case. The upper left shows an unaccelerated gold standard, the upper middle shows a 2×4-fold accelerated SENSE reconstruction, the upper right shows a SENSE g-factor noise map, the lower left shows a low resolution reference image, the lower middle shows an LSNR reconstruction, and the lower right shows an LSNR g-factor noise map.

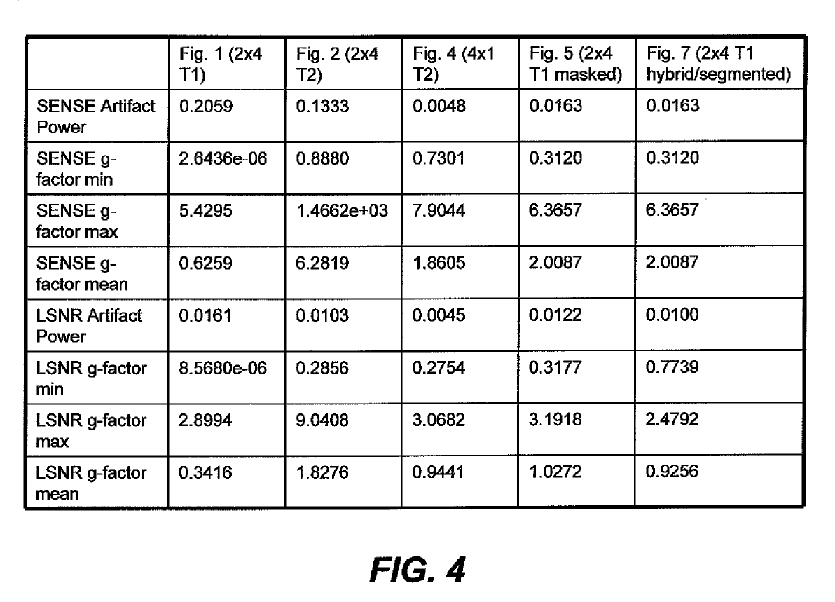

To demonstrate the results quantitatively, AP and g-factor values are calculated for several experiments and presented in the table in FIG. 4. A significant decrease in g-factor noise and AP for LSNR images are demonstrated in comparison with original SENSE reconstruction.

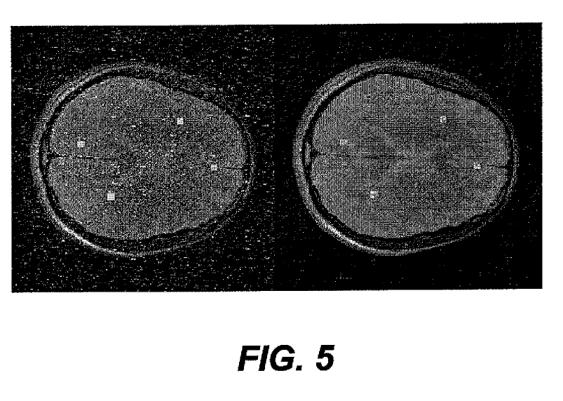

FIG. 5 illustrates four discrete artificial lesions added to the SENSE reconstructed image of FIG. 3, without adding lesions to the reference image, with the corresponding LSNR reconstruction. The image on the left in FIG. 5 shows a 2×4-fold accelerated SENSE reconstruction with 4 lesions added, and the image on the right shows an LSNR reconstruction. The resulting LSNR image preserves the lesions without creating any significantly new visually appreciable artifacts. Results using LSNR are comparable for both T₁- and T₂-weighted images as expected. LSNR is also successful when using a reference image slightly displaced from the image slice. This suggests that the LSNR algorithm may not directly replicate prior knowledge.

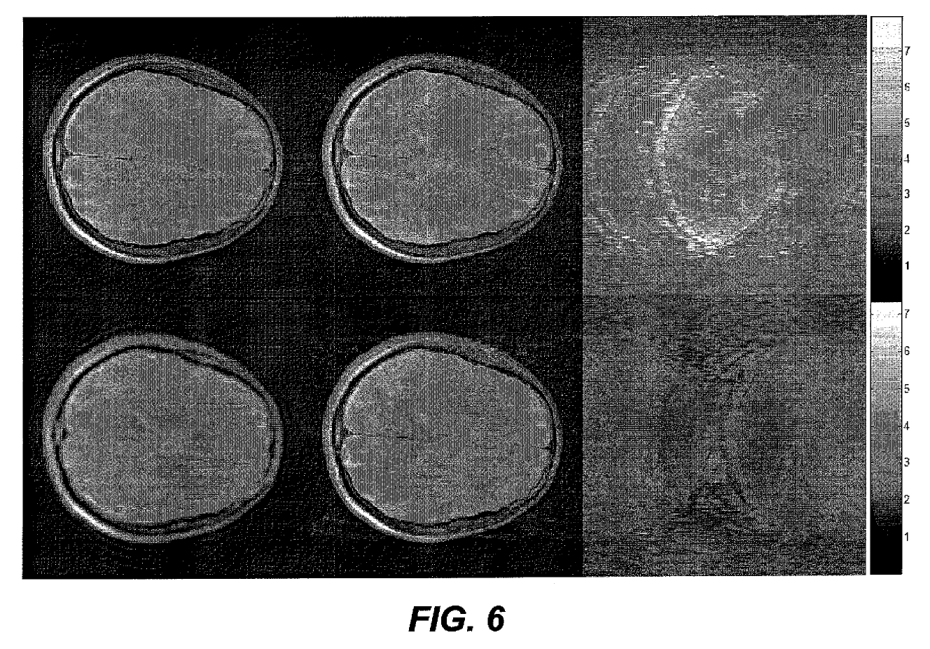

LSNR techniques were also studied in experiments involving submaximally accelerated reconstructions. FIG. 6 illustrates images wherein a 4-fold accelerated reconstruction was performed using a T2-weighted in vivo brain image obtained with an 8 element head array. The upper left shows an unaccelerated gold standard, the upper middle shows 4-fold accelerated SENSE reconstruction, the upper right shows a SENSE g-factor noise map, the lower left shows a low resolution reference image, the lower middle shows an LSNR reconstruction, and the lower right shows an LSNR g-factor noise map. It should be noted that the 4-fold SENSE image is significantly less noisy than the SENSE image resulting from the 8-fold reconstruction. AP and g-factor values associated with FIG. 6 are presented in the table in FIG. 4, and show that g-factor noise is in fact removed in the tissue regions. Furthermore, the AP value for LSNR, at 0.0045, is approximately equal to the AP for SENSE, at 0.0048.

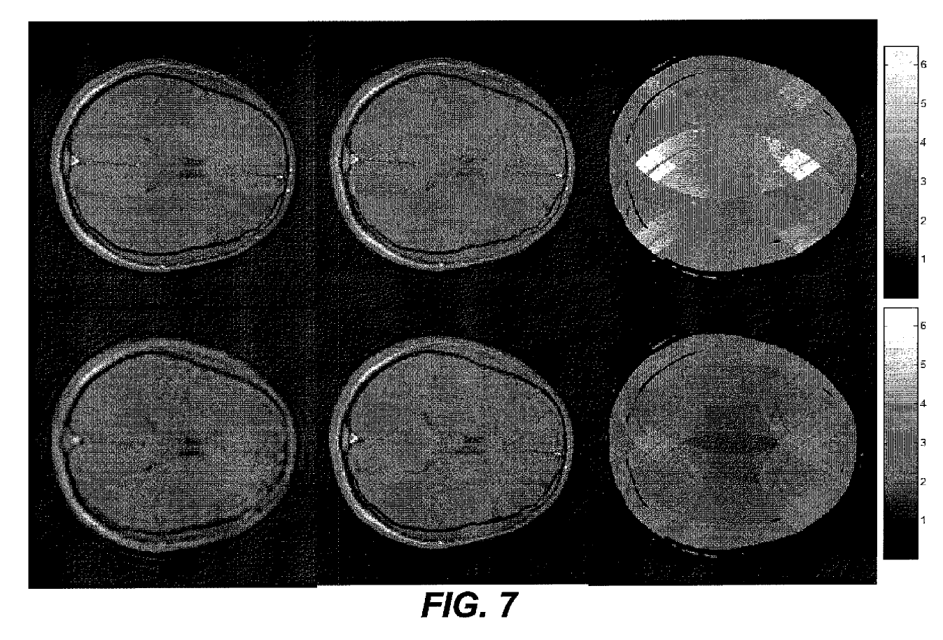

A similar result is demonstrated in FIG. 7, except that in this case the SENSE image is reconstructed at 8×after its corresponding coil sensitivity maps were masked by determining the set of pixels that are less than 5% of the maximum reference image pixel intensity and by setting corresponding indices of the coil sensitivities to zero. The upper left shows the unaccelerated gold standard, the upper middle shows a 2×4-fold accelerated masked SENSE reconstruction, the upper right shows a SENSE g-factor noise map, the lower left shows a low resolution reference image, the lower middle shows an LSNR reconstruction, and the lower right shows an LSNR g-factor noise map. The SENSE image is markedly reduced in noise compared to its unmasked counterpart, and LSNR is again successful in reducing AP and g-factor noise (see FIG. 4).

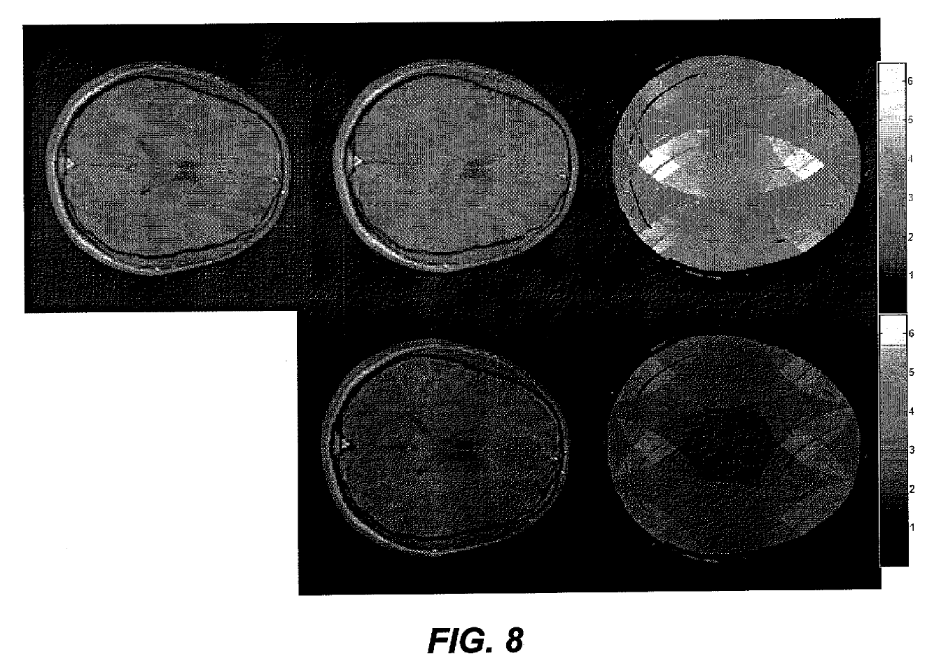

FIG. 8 shows images obtained using a post-processing technique that removes PCT from a masked 8-fold accelerated SENSE reconstruction. The PCT portion of an image usually has relative high intensity, and is adjacent to areas of relative low intensity. By first removing the PCT, the LSNR method may be applied only on the brain tissue. The PCT may then be added back to the resulting LSNR image, forming an LNSR hybrid image. The upper left image in FIG. 8 shows the unaccelerated gold standard, the upper middle shows a 2×4-fold masked SENSE reconstruction from FIG. 7, the upper right shows a SENSE g-factor noise map from FIG. 7, the lower left shows an LSNR hybrid reconstruction, where the pericalvarial tissue is from the masked SENSE image, while the remaining parenchyma is derived from an LSNR reconstruction, and the lower right shows an LSNR hybrid g-factor noise map.

AP and g-factor values associated with FIG. 8 are presented in the table in FIG. 4. These values, along with images in FIG. 8, show that an LSNR hybrid image may have significantly reduced artifact and g-factor noise, when compared to the LSNR and SENSE images in FIG. 7. Additionally, based on visual comparison focusing on clarity of critical brain structures such as insular cortex, white matter corona radiata, the Sylvian fissure, and other sulci, an LSNR hybrid image may have improved overall quality compared to the masked SENSE and LSNR images in FIG. 7. This suggests that predominant artifacts in LSNR reconstruction may correspond to edge artifacts related to areas of higher intensity, in this case the PCT, and that artifacts in LSNR reconstruction may be reduced by a hybrid reconstruction technique such as the one described herein.

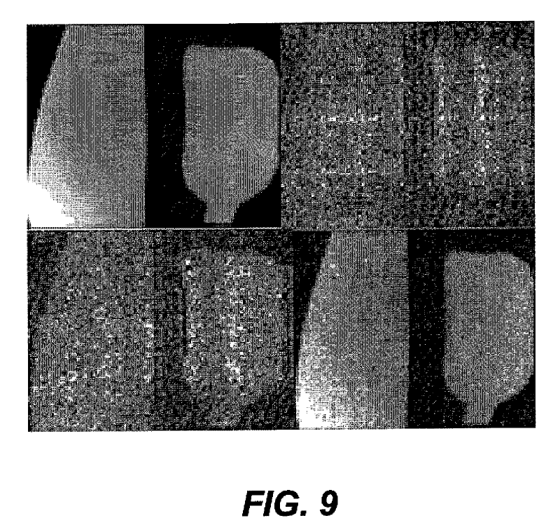

FIG. 9 illustrates maximal 8×4 accelerated reconstructions performed on T2-weighted phantom images acquired from a 32 element body array. The upper left shows the unaccelerated gold standard, the upper right shows a 32×(8×4) SENSE reconstruction, the lower left shows a 1-component least squares noise reduction, and the lower right shows a 9-component least-squares noise reduction. Using a low resolution reference (not shown), the LSNR technique may significantly reduce noise from the SENSE reconstruction. Furthermore, multiple singular values (in particular, up to 9 singular values) are used in this experiment. By contrast, only one singular value is used in the previous experiments. The empirical findings suggest that using approximately 40% of singular values may offer a good balance between noise reduction and inclusion of prior information in a final image.

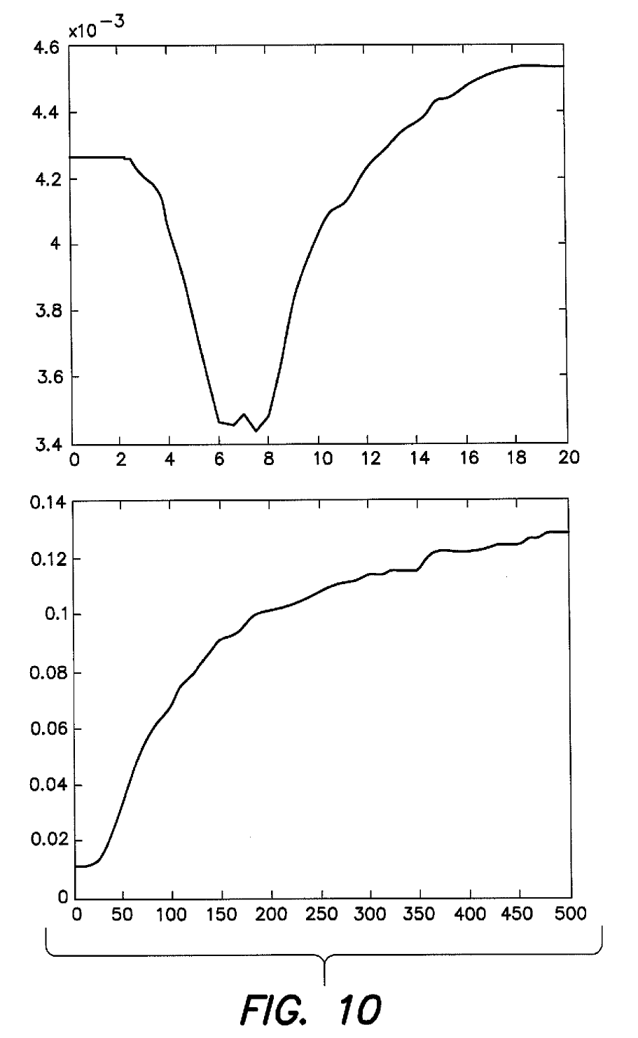

In FIG. 10, AP is directly plotted against condition number threshold (CNT). For 4×reconstructions (shown on the top), AP reaches minimum at a nonzero CNT value, roughly 7.5, and the minimum AP represents a 24% improvement over the SENSE image. For 8×reconstructions (shown on the bottom), AP reaches a minimum at CNT=0, suggesting that all voxels may benefit from the LSNR method since they were all poorly conditioned. The minimum AP represents a 91% improvement over the SENSE image.

The above-described embodiments of the invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

It should be appreciated that the various methods outlined herein may be coded as software that is executable on one or more processors that employ any one of a variety of operating systems or platforms. Additionally, such software may be written using any of a number of suitable programming languages and/or conventional programming or scripting tools, and also may be compiled as executable machine language code. In this respect, it should be appreciated that one embodiment of the invention is directed to a computer-readable medium or multiple computer-readable media (e.g., a computer memory, one or more floppy disks, compact disks, optical disks, magnetic tapes, etc.) encoded with one or more programs that, when executed, on one or more computers or other processors, perform methods that implement the various embodiments of the invention discussed above. The computer-readable medium or media can be transportable, such that the program or programs stored thereon can be loaded onto one or more different computers or other processors to implement various aspects of the invention as discussed above.

It should be understood that the term “program” is used herein in a generic sense to refer to any type of computer code or set of instructions that can be employed to program a computer or other processor to implement various aspects of the invention as discussed above. Additionally, it should be appreciated that according to one aspect of this embodiment, one or more computer programs that, when executed, perform methods of the invention need not reside on a single computer or processor, but may be distributed in a modular fashion amongst a number of different computers or processors to implement various aspects of the invention.

Various aspects of the invention may be used alone, in combination, or in a variety of arrangements not specifically discussed in the embodiments described in the foregoing, and the aspects of the invention described herein are not limited in their application to the details and arrangements of components set forth in the foregoing description or illustrated in the drawings. The aspects of the invention are capable of other embodiments and of being practiced or of being carried out in various ways. Various aspects of the invention may be implemented in connection with any type MR imaging equipment of any configuration. No limitations are placed on scanner implementation. Accordingly, the foregoing description and drawings are by way of example only.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

What is claimed is:

1. A method for noise reduction in parallel magnetic resonance (MR) imaging using a plurality of radio frequency (RF) coils, the method comprising:
performing a first MR scan of a target to obtain first MR data;
performing a second MR scan of the target to obtain second MR data, the second MR data obtained from operating the plurality of coils substantially in parallel; and
directly computing a noise estimate associated with the second MR data based at least in part on the first and second MR data.

2. The method of claim 1, further comprising:
obtaining a noise-reduced image based at least in part on the second MR data and the noise estimate.

3. The method of claim 1, wherein the noise estimate is computed at least in part by solving one or more algebraic equations.

4. The method of claim 3, wherein the one or more algebraic equations are obtained at least in part by substituting information derived from the first MR data for information approximating noiseless MR data, wherein the noiseless MR data are unknown.

5. The method of claim 3, wherein the one or more algebraic equations are solved using a least squares technique.

6. The method of claim 1, further comprising:
reconstructing a first image based at least in part on the first MR data;
reconstructing a second image based at least in part on the second MR data,
wherein the noise estimate is computed based at least in part on a difference between the first and second images.

7. The method of claim 6, wherein the second image is reconstructed based on the second MR data and a sensitivity matrix associated with a geometric configuration of the plurality of RF coils.

8. The method of claim 7, wherein the sensitivity matrix is decomposed using singular value decomposition.

9. The method of claim 1, wherein the first MR data is obtained at low resolution relative to the second MR data.

10. The method of claim 1, wherein the plurality of RF coils includes N RF coils, and wherein an image acquisition acceleration factor K achieved using the plurality of RF coils is close to or equal to N.

11. A computer storage device encoded with a program for execution on at least one processor, the program when executed performs a method for noise reduction in parallel magnetic resonance (MR) imaging, the method comprising acts of:
receiving first MR data;
receiving second MR data obtained from operating a plurality of RF coils substantially in parallel;
directly computing a noise estimate associated with the second MR data based at least in part on the first and second MR data; and
computing a noise-reduced image based at least in part on the second MR data and the noise estimate.

12. The computer storage device of claim 11, wherein computing the noise estimate includes solving one or more algebraic equations obtained at least in part by substituting information derived from the first MR data for information associated with unknown noiseless MR data.

13. The computer storage device of claim 11, wherein the one or more algebraic equations are solved using a least squares technique.

14. The computer storage device of claim 11, wherein the method further comprises acts of:
reconstructing a first image based at least in part on the first MR data; and
reconstructing a second image based at least in part on the second MR data,
wherein the noise estimate is computed based at least in part on a difference between the first and second images.

15. The computer storage device of claim 14, wherein the second image is reconstructed based on the second MR data and a sensitivity matrix associated with a geometric configuration of the plurality of RF coils.

16. A system for performing parallel magnetic resonance (MR) imaging comprising:
a scanning apparatus comprising a plurality of radio frequency (RF) coils, the scanning apparatus configured to perform accelerated image acquisition by operating the plurality of coils substantially in parallel to obtain parallel MR data; and
at least one signal processing device adapted to receive the parallel MR data, the at least one signal processing device configured to reduce acceleration-related noise in the parallel MR data by receiving reference MR data, directly computing a noise estimate associated with the parallel MR data based at least in part on the parallel MR data and the reference MR data, and generating at least one noise-reduced MR image based at least in part on the accelerated MR data and the noise estimate.

17. The system of claim 16, wherein the signal processing device computes the noise estimate at least in part by solving one or more algebraic equations that are obtained at least in part by substituting information derived from the reference MR data for information associated with unknown noiseless MR data.

18. The system of claim 17, wherein the one or more algebraic equations are solved using a least squares technique.

19. The system of claim 16, wherein the reference MR data is obtained at low resolution relative to the parallel MR data.

20. The system of claim 20, wherein the scanning apparatus includes N coils, and wherein the scanning apparatus is configured to obtain MR data at an image acquisition acceleration factor K that is close or equal to N.
